(19) United States
(12) Patent Application Publication
(10) Pub. No.: US 2003/0180710 A1
(43) Pub. Date: Sep. 25, 2003

(54) Method of enhancing the efficiency of a pharmaceutical business

(76) Inventors: Wai-Nang Paul Lee, Palos Verdes Estate, CA (US); Laszlo G. Boros, Torrance, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/192,743

(22) Filed: Jul. 9, 2002

Related U.S. Application Data
(60) Provisional application No. 60/367,142, filed on Mar. 22, 2002.

(51) Int. Cl. G06F 17/60; C12Q 1/00; G06F 19/00; G01N 33/48; G01N 33/50

(52) U.S. Cl. 435/4; 702/19; 705/2

(57) ABSTRACT

A method of doing business is disclosed whereby the process for selecting drug candidates is improved. The method involves the application of a technology which makes it possible to determine metabolic processes involved in the formation of any glucose-based metabolite. A precursor molecule is labeled with a stable carbon (¹³C) isotope at specific positions. The label is allowed to distribute and rearrange in the system. Metabolites are recovered and analyzed against a control system or known biochemical reactions and/or cycles to determine information such as metabolic pathway substrate flux caused by a compound acting on the system.

### CROSS-REFERENCE

This application claims the benefit of U.S. Provisional Application No. 60/367,142, filed Mar. 22, 2002, which application is incorporated herein by reference.

### GOVERNMENT RIGHTS

This invention was made with government support under federal grant PO 1 CA42710-15 awarded by the National Institutes of Health to the University of California at Los Angeles (UCLA) Clinical Nutrition Research Unite (CNRU). The United States Government may have certain rights in this invention. This grant was awarded based on a competitive peer review in order to support academic research in the stable isotope core laboratory of this CNRU.

### FIELD OF THE INVENTION

This invention relates generally to a method of doing business which method improves the drug discovery and drug testing processes, for example, by applying biochemical methodologies and in particular by using an isotope such as a stable (¹³C) isotope for labeling a metabolome to examine mechanisms of cellular substrate flow modifications in response to various drugs, food additives, natural compounds and environmental factors, in order to reveal how they affect cellular physiology, phenotype and function, based on metabolic pathway substrate flow distribution, intermediate production and end-product synthesis.

### BACKGROUND OF THE INVENTION

The identification of the biological pathway of action of a drug or drug candidate is a problem of great commercial and human importance. Although the primary molecular target of and cellular pathways affected by a drug are often known or suspected because the drug was originally selected by a specific drug screen, it is important to verify its action on such a primary pathway and to quantify its action along other secondary pathways which may be harmful, or may be beneficial, often in unsuspected ways. In other cases, the primary pathways of drug action are unknown, and these must be determined.

This information is important in many areas of practical research, such as, for example, drug discovery, which is a process by which bioactive compounds are identified and preliminarily characterized. Drug discovery is a critical step in the development of treatments for human diseases. There are different approaches used by companies in their search for new drugs.

One approach begins with a screen for compounds that have a desired effect on a cell (e.g., induction of apoptosis), or organism (e.g., inhibition of angiogenesis) as measured in a specific biological assay. Compounds with the desired activity may then be modified to increase potency, stability, or other properties, and the modified compounds retested in the assay. Thus, a compound that acts as an inhibitor of angiogenesis when tested in a mouse tumor model may be identified, and structurally related compounds synthesized and tested in the same assay. One limitation of this approach is that, often, the mechanisms of action, such as the molecular target(s) and cellular pathways affected by the compound, are unknown, and cannot be determined by the screen. In addition, the assay may provide little information about the specificity and toxicity, either in terms of targets or pathways, of the drug's effect. Finally, the number of compounds that can be screened by assaying biological effects on cells or animals is limited by the required experimental efforts.

Another approach to drug screening involves testing numerous compounds for a specific effect on a known molecular target, typically a cloned gene sequence or an isolated enzyme or protein. For example, high-throughput assays can be developed in which numerous compounds can be tested for the ability to change the level of transcription from a specific promoter or the binding of identified proteins. Although the use of high-throughput screens is a powerful methodology for identifying drug candidates, it has limitations. A major drawback is that the assay provides little or no information about the effects of a compound at the cellular or organismal level, in particular information concerning the actual cellular pathways affected. These effects must be tested by using the drug in a series of cell biologic and whole animal studies to determine toxicity or side effects in vivo. In fact, analysis of the specificity and toxicity studies of candidate drugs can consume a significant fraction of the drug development process (see, e.g., Oliff et al., 1997, “Molecular Targets for Drug Development,” in DeVita et al. Cancer: Principles & Practice of Oncology 5th Ed. 1997 Lippincott-Raven Publishers, Philadelphia).

Several gene expression assays are now becoming practicable for quantitating the drug effect on a large fraction of the genes and proteins in a cell culture (see, e.g., Schena et al, 1995, Quantitative monitoring of gene expression patterns with a complementary DNA micro-array, Science 270:467-470; Lockhort et al., 1996, Expression monitoring by hybridization to high-density oligonucleotide arrays, Nature Biotechnology 14:1675-1680; Blanchard et al., 1996, Sequence to array: Probing the genome's secrets, Nature Biotechnology 14, 1649; 1996, U.S. Pat. No. 5,569,588, issued Oct. 29, 1996 to Ashby et al. entitled “Methods for Drug Screening”). Raw data from these gene expression assays are often difficult to coherently interpret. Such measurement technologies typically return numerous genes with altered expression in response to a drug, typically 50-100, possibly up to 1,000 or as few as 10. In the typical case, without more analysis, it is not possible to discern cause and effect from such data alone. The fact that one or a few genes among many has an altered expression in a pair of related biological states yields little or no insight into what caused this change and what the effects of this change are. These data in themselves do not inform an investigator about the pathways affected or mechanism of action. They do not indicate which effects result from effects on a primary pathway versus which effects are the result of other secondary pathways affected by the drug. Knowledge of all these affected pathways individually is useful in understanding efficacy, side-effects, toxicities, possible failures of efficacy, activation of metabolic responses, and so forth. Further, identification of all pathways of drug action can lead to discovery of alternate pathways suitable to achieve the original therapeutic purpose.

Without effective methods of analysis, one is left to ad hoc further experimentation to interpret such gene expression results in terms of biological pathways and mechanisms. Systematic procedures for guiding the interpretation of such data and such further experimentation, at least in the case of drug target screening, are needed.

One approach to identify pathways of drug action is presented in U.S. Pat. No. 5,965,352. A method of evaluating the effects of a drug in a multiple dose clinical trial is presented in U.S. Pat. No. 6,041,788. A method for determining the presence of a number of primary targets of a drug is present in U.S. Pat. No. 6,146,830. A computer system for determining primary targets for a drug is presented in U.S. Pat. No. 6,300,078. In addition to these generalized methods these exit various methods of metabolic profiling.

Metabolic profiling or metabolomics is an old investigative field where the amounts or concentrations of various metabolites of various pathways in living organisms are measured and, from these determinations, activities of the respective metabolic pathways are predicted (Katz, J., Rognstad, R. (1967). Specific examples include the labeling of pentose phosphate from glucose-¹⁴C and estimation of the rates of transaldolase, transketolase, the contribution of the pentose cycle to ribose phosphate synthesis. Biochemistry 6: 2227-47). In general, these techniques only provide information on a static picture of a cell at one point in time and only measure synthesis rates without being able to reveal specific reactions and their contributions to end-product synthesis. The technique does not exactly reveal the previous metabolic steps and the exact synthesis pathways but only estimates the involvements of possible metabolic pathways based on existing biochemical information.

There are many alternative pathways throughout cellular metabolism to produce various metabolites, therefore, it is often difficult, if not impossible, to elucidate particular enzymatic reactions using static metabolic profiling and thus taking “metabolic snapshots” (Raamsdonk, L. M., Teusink, B., Broadhurst, D., Zhang, N., Hayes, A., Walsh, M. C., Berden, J. A., Brindle, K. M., Kell, D. B., Rowland, J. J., Westerhoff, H. V., van Dam, K., Oliver, S. G. (2001). A functional genomics strategy that uses metabolome data merely to reveal the phenotype of silent mutations Nat Biotechnol 19: 45-50) can not reveal substrate flow and enzymatic substrate modifications in interconnected and complex metabolite networks.

Leading laboratories in stable isotope based metabolite research use single labeling patterns and measure single pathways in mammalian cells in order to reveal specific synthesis steps of a few pre-selected bio-molecules. These pathways may be involved in cell proliferation (Neese, R. A., Siler, S. Q., Cesar, D., Antelo, F., Lee, D., Misell, L., Patel, K., Tehrani, S., Shah, P., Hellerstein, M. K. (2001). Advances in the stable isotope-mass spectrometric measurement of DNA synthesis and cell proliferation have also been described Anal Biochem 298: 189-95), however the method only measures new cell production through DNA synthesis without the specifics of metabolic pathway activities and their contribution to the cellular proliferation process. The stable isotope labeling technique can be applied to in vivo in animal experiments or even in human studies without potential harm to the subject while providing information such as in standard cell counting techniques, BrDU labeling or ³H-thymidine incorporation into the DNA of cultured cells. Further, others have carried out work applied to gluconeogenesis (Previs, S. F., Brunengraber, H. (1998) to measure the production of glucose in vivo (Curr Opin Clin Nutr Metab Care 1: 461-5), as well as de novo lipid and fatty acid synthesis (Verhoeven, N. M., Schor, D. S., Previs, S. F., Brunengraber, H., Jakobs, C. (1997). Stable isotope studies of phytanic acid alpha-oxidation and in vivo production of formic acid has also been described (Eur J Pediatr 56: S83-7). Stable isotopes are also used as standards for quantification of known compounds in the blood and body fluids (Leis, H. J., Windischhofer, W., Raspotnig, G., Fauler, G. (2001) and others have described stable isotope dilution negative ion chemical ionization gas chromatography-mass spectrometry for the quantitative analysis of paroxetine in human plasma (J Mass Spectrom 36: 923-8; Andrew, R. (2001) as well as the clinical measurement of steroid metabolism (Best Pract Res Clin Endocrinol Metab 15: 1-16). Although important for the quantitation of metabolite synthesis and turnover rates, these papers reveal no attempt to analyze the metabolome, as a whole, by its selected and representative components synthesized through individual metabolic reactions, which are linked, interconnected and are capable of cross-label the cellular intermediary metabolite pool as they rearrange and re-distribute ¹³C labeled substrate carbons from one stable isotope labeled precursor, which, in turn, imprints a metabolic “history” and “memory” into the dynamically formed product pool throughout the life cycle of the organism and drug treatments (FIG. 1).

The present invention provides a new method of doing business whereby the specific cellular metabolic effects of new lead compounds such as drug candidates can be tracked through the stable isotope labeled metabolome. Information obtained is used to enhance the process by which compounds such as drugs are selected, developed and marketed. Thus, the invention can provide drug companies with more comprehensive, specific information on a drug's mechanism of action revealing information for drug approval agencies and clinical investigators. Thus, the method endeavors to decrease research and developmental costs and thereby increase profits and the number of compounds such as drugs which are commercially marketed for the good of mankind.

### SUMMARY OF THE INVENTION

A method of doing business is disclosed and described which method enhances the ability of organizations to identify the safety, efficacy, mechanisms of action and/or other information about a compound being tested. The method can involve (a) profiling a compound or group of individual compounds to determine information such as how a drug candidate effects a cellular system on a molecular metabolic level, (b) selecting a compound for further study based on the profile obtained, (c) carrying out clinical trials on the selected compound to obtain additional information and regulatory approval to sell the drug, and (d) selling the drug or compound by itself or in an appropriate formulation.

The step of profiling the compound (e.g. drug candidate) may be carried out using a stable ¹³C isotope based glucose substrate which may be [1,2-¹³C₂]glucose. The isotope labeled substrate can readily and dynamically label molecules involved in intracellular metabolic pathways and specific active metabolic steps. Molecules will incorporate the ¹³C label in a specific manner which provides a stable isotope enriched metabolome in the form of substrates and products, which reveal synthesis patterns, destinies and distributions of the labeled molecule (such as the labeled glucose) among major metabolic pathways broadly. This precise labeling and molecule tracking technique may be utilized for drug target discovery, and for testing and screening of compounds, which may be used as pharmaceutically active drugs, food additives, natural products with physiological activities or changes in cellular environment. The metabolic effects of new compounds such as drugs, as specifically tested by ¹³C labeled glucose, reveal the metabolic end result of genetic manipulations and cell-signaling events because changes in the stable isotope labeled metabolome closely reflect changes in metabolic enzyme activities that are primarily controlled by genes or protein phosphorylating signals. Metabolic substrate flow changes are a critical part of the metabolic adaptation process of mammalian cells to growth modifying signaling or genetic events and the invention can reveal such changes. Metabolic adaptation is also required to change cellular phenotypes and function, which can also be mechanistically characterized by the invention. This is because the metabolome serves as the ultimate and last resort of implementing profound changes in cellular function in a chain of genetic, proteomic and metabolic events in any living organism. As the genome and proteome already have their own labeling technologies and techniques, it is evident that comprehensive studies of the metabolome will also require a broad, effective, yet specific label system for drug studies to come. Thus, a gap in human experimental capacity to study cellular metabolism as part of the genetic-proteomic-metabolimic chain of events may be filled with the invention.

A labeled substrate molecule such as ¹³C labeled glucose substrate is provided to a system which may be cells in a cell culture. The cells are used to create an information profile which details any desired aspect(s) of cellular metabolism including metabolic pathway substrate flow, specific metabolite synthesis patterns, rate of metabolite synthesis, contribution of individual synthetic reactions, etc. Once the information profile is created labeled glucose can be added to a substantially identical system to which is added a compound such as a drug to be tested. The information profile created in the absence of the drug is then compared to a new information profile created with the drug in the system. Other parameters such as the concentration of the drug added to the system and/or the amount of time allowed to pass can be changed to obtain different information profiles which when analyzed provide information on how the drug effects the system on a molecular metabolic level. This allows the testing of new drug candidates in a dose and time dependent fashion with great specifics regarding their metabolic effects, toxicity and regulatory mechanisms on metabolic pathway substrate flow, cell function and phenotype. The data of the information profiles can be analyzed in any desired manner, e.g. by visual comparisons and/or by the use of appropriate computer software. Based on the drug action on the metabolome further studies can be initiated to measure metabolic enzyme levels, the synthesis of metabolic enzyme proteins and the expression of metabolic enzymes as biomarkers for disease processes or drug actions as shown in FIG. 2. The invention can be used to show how the metabolic effect of new drug candidates correlate with changes in the genome and proteome. Drug candidates which demonstrate the desired profile are tested further such as in clinical trials. Drugs which are successful in the clinical trials, e.g. are shown to be safe and effective, are marketed with appropriate government approval.

The invention provides a new use for the non-toxic, stable ¹³C labeled glucose isotope. The methodology is applied for characterizing the complex dynamic metabolic profiles of diseases and to investigate the mechanism of action of new and existing compounds and, particularly, the action of putative therapeutic compounds. The invention enhances the ability for discovering new drug target sites through metabolic enzymes, which strongly and effectively control substrate flow and distribution in living organisms. This technique can also be used in the drug industry to reveal the exact mechanism of action of new drugs on metabolism and to reveal toxicity, which will accelerate the drug testing, candidate selection and drug approval processes. By enhancing the efficiency of these various processes the efficiency of the overall method of doing business is enhanced.

Another aspect of the invention is a marketed drug sold with a written label which describes the drug's mechanism of action. The ¹³C labeling methodology described here is capable of providing specific information on precisely how the drug effects a particular biochemical reaction. Thus, drugs are provided with a written description on how the drug acts and will be more accepted by both the medical community and patients wishing to make more informed decisions about their treatment. The written label sold with the drug in commerce may be as simple as an indication that the drug was developed, at least in part, using ¹³C labeling methodology or using [1,2-¹³C₂]glucose labeling methodology.

The understanding of drug actions and the mechanisms of diseases require characterization of metabolic pathways through the flow of substrates. Genes and signal transduction pathways can only trigger changes in cellular metabolic activity but they can not reveal if metabolic enzymes are activated and their substrates abundantly present. The present invention provides a stable isotope based dynamic metabolic profiling system with the purpose of obtaining dynamic metabolic substrate flow information through the pentose cycle, glycogen synthesis, tricarboxylic acid cycle, glycolysis, lactate synthesis, glutamate production, fatty acid synthesis and nucleic acid ribose and deoxyribose synthesis pathways by themselves or simultaneously. It is therefore a comprehensive and dynamic technique based on precisely directed isotope labeling that can reveal specific metabolic pathway flux changes in disease and health. Further, the invention can be used to reveal the metabolic mechanisms of drug actions and that of natural/synthetic compounds in various disease treatment modalities and to improve metabolic engineering.

Aspects of this invention may include:

1) The ¹³C stable isotope labeled metabolome (e.g. [1,2-¹³C₂]glucose) which allows not only the determination of substrate levels but also the determination of through which steps molecular synthesis pathways are linked;

2) A number of metabolic processes may be simultaneously determined in the same cell system or organism;

3) The preferred synthesis steps that can be effectively targeted by new drugs (steps with large control coefficients) for individual metabolites may be predicted and determined;

4) The obtained metabolic profiles of diseases or drug actions may be compared, correlated and used to define disease states, and responses to gene manipulations, signaling events or drug treatments;

5) Early toxic effects of new compounds may be readily revealed by the deterioration of isotope labeled carbon flow through life sustaining metabolic pathways; and

6) The direct metabolic effects of “silent genes”, which do not alter metabolite concentrations but synthesis pathways only for the same metabolite may be revealed.

The method of doing business of the invention uses a dynamic metabolic profiling method that involves a “smartly” labeled glucose substrate for the positional labeling of other metabolites in the cell during various metabolic steps. By analyzing the stable isotope labeled metabolome (e.g. [1,2-¹³C₂]glucose) the invention may reveal synthesis pathway specific metabolic adaptive changes in response to practically any condition in the environment, during health, disease or drug treatments. The dynamic and comprehensive stable isotope based metabolic profiling technique may utilize a broad yet specific approach for multiple pathway flux measurements and synthesis pathway activities based on the “smartly” labeled, non-toxic and stable isotope tracer. This specifically labeled substrate introduces “heavy” non-radiating carbons into specific positions of the carbon chain of several key intermediates, which then reveal vital information about pathway substrate flux and re-distribution after recovery of the label from the product bio-molecules of the metabolome. The invention makes it possible to determine the concentrations of intermediate molecules as well as the ability to determine the dynamics of synthesis and turnover rates with accurate details of the contribution of specific synthetic reactions across metabolic networks.

The invention provides a method for solving the basic problem of investigating metabolism in a dynamic, comprehensive and specific manner which can reveal actual metabolic pathway substrate utilization and distribution patterns, predict changes in metabolic enzyme activities and determine the metabolic end result of various genetic mutations, silent genes, disease processes, cell signaling events and chemical drug interventions.

The methodology of the invention may possess the advantage of being an interactive, comprehensive and treatment responsive metabolic screening tool for the drug industry and academic investigative processes.

The methodology of the invention may use isotope incorporation data to measure synthesis rates and molecule turnover rates while also providing the specificity of identifying particular pathways and making it possible to determine their contributions to previous and subsequent metabolic steps. The invention can make it possible to provide drug testing, drug target discovery or drug screening and apply the technology to studying basic biochemical events in primitive species including bacteria and yeast.

The present invention comprises the use of the stable [1,2-¹³C₂]glucose tracer for metabolic pathway analyses in cultures of mammalian cells, bacteria, virus hosting cells, phage hosting bacteria, tissue slices, perfused organs, living animals or humans. The method further comprises applying such to individual pathway flux measurements, and analyzing the metabolome as whole or testing drugs. The invention provides a dynamic and comprehensive metabolic profiling technique by using specifically labeled glucose substrate isotopes which not only turn into intermediary metabolites but also produce mass isotopomers of these metabolites which can be separated, measured and quantitated using liquid and gas chromatography separation and mass spectrometry (GC/MS), other mass spectrometric analysis or nuclear magnetic resonance (NMR) instruments.

Preparation of samples for dynamic metabolic profiling is the same of what have been described in the medical literature for previous studies using less effective label systems. The major development in the invention is the design of the label system, its distribution and recovery from the same metabolites that have previously been isolated for metabolic profiling studies. One major difference however is that there may be numerous metabolites isolated from the cell culture media, cell pellets or blood plasma simultaneously. This way the invention makes it possible to measure interconnected metabolic pathway carbon substrate flow using one common substrate, glucose, for metabolic profiling drug action studies.

After treatments while incubating with the isotope labeled glucose substrate, the profiling study may begin with the separation of cell pellets, cell culture media, blood plasma/serum or body fluids. The cell culture media is then used for lactate and glutamate analyses. Lactate is an abundant cell media component of the three carbon metabolite pool of the cell and it is used to determine specifically the relative activity of pentose cycle glucose oxidation and recycling into glycolysis as the per cent of substrate flux through glycolysis, also known as the Embden-Meyerhoff-Pamas pathway. Glutamate label accumulation represents tricarboxylic acid (TCA) cycle carbon substrate flow. Nucleic acid ribose and deoxyribose are used to determine cell viability (ribose) and cell proliferation (deoxyribose). Cell cycle progression and the frequency of cell divisions are determined by the accumulation of ¹³C label into deoxyribose, while cell viability, apoptosis and necrosis are determined by the differential incorporation of ¹³C into ribose and deoxyribose. An aspect of the label system of the invention is that it can differentiate between glucose oxidation and nonoxidative ribose/deoxyribose synthesis during nucleic acid production in disease and health as well as during drug testing. Glycogen glucose represents glycogen synthesis, which can originate from phosphorylated (activated) glucose (direct glycogen synthesis) or indirectly from pentoses after direct glucose oxidation in the pentose cycle. Non-essential fatty acids of the saturated and desaturated kinds indicate the rate of de novo fatty acid synthesis from glucose and the contribution of fatty acid synthase, chain elongase and desaturase to cell differentiation, hormone synthesis and drug effects.

The label system of the invention can clearly differentiate and characterize these pathways and their responses to drug treatments in a specific and effective manner in a simple series of labeling and drug treatment studies. The stable isotope label system does not interfere with drug effects and the effect of drugs can therefore be studied simultaneously in virtually all major interconnected metabolic pathways which also serve in energy production, cell proliferation, enzyme, hormone or specific metabolite synthesis pathways.

Although there are many tools and devices to analyze stable isotope labeled metabolites that include NMR or new mass spectrometry techniques, such as MALDI-TOF, which require different sample preparation methods and techniques, it needs to be pointed out that [1,2-¹³C₂]glucose provides a comprehensive, effective and cost efficient label design for comprehensive dynamic metabolic profiling purposes.

The invention makes it possible to track enzymatic modifications of the precisely labeled precursor molecule as it makes specific rearrangements in the positions and amounts of the stable isotope incorporated into subsequent bio-molecules throughout the metabolome. The rearrangements of labeled carbons within a molecule yield the dynamic history of that molecule from the precursor to the product and between, much the same way as if the labeled molecule had a “memory” imprinted with the steps it went through.

These and other objects, advantages, and features of the invention will become apparent to those persons skilled in the art upon reading the details of the invention as more fully described below.

### BRIEF DESCRIPTION OF THE DRAWINGS

FIG. 1 is a schematic drawing of the overall metabolic networks of living organisms, the inter-connecting metabolic steps and key metabolites that are readily labeled by [1,2-¹³C₂]glucose as the tracer precursor.

FIG. 2 is the schematic drawing of a stable isotope labeling experiment.

FIG. 3 is a schematic drawing of a structure of a preferred embodiment of a labeled glucose molecule along with possible rearrangements of ¹³C in various metabolites of glycolysis using [1,2-¹³C₂]glucose as a single tracer.

FIG. 4 is a schematic drawing of structures of labeled compounds involved in the formation of [2,3-¹³C₂]lactate through the Embden-Meyerhoff-Pamas pathway.

FIG. 5 is a schematic drawing of structures of compounds involved in the rearrangement of ¹³C in pentose cycle metabolites due to direct glucose oxidation.

FIG. 6 is a schematic drawing of structures of compounds involved in formation of [1-¹³C]ribose-5P in the non-oxidative pentose cycle after glucose oxidation.

FIG. 7 is a schematic drawing of structures of compounds involved in the formation of [1,2-¹³C₂]ribose through the non-oxidative reactions of the pentose cycle.

FIG. 8 is a schematic drawing of structures of compounds involved in the formation of ¹³C labeled acetyl-CoA and glutamate through pyruvate dehydrogenase and pyruvate carboxylase in the TCA cycle.

### DETAILED DESCRIPTION OF PREFERRED EMBODIMENTS

Before the present invention is described, it is to be understood that this invention is not limited to molecules and specific method steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a labeled molecule” includes a plurality of such labeled molecules and reference to “the step” includes reference to one or more steps and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Invention in General

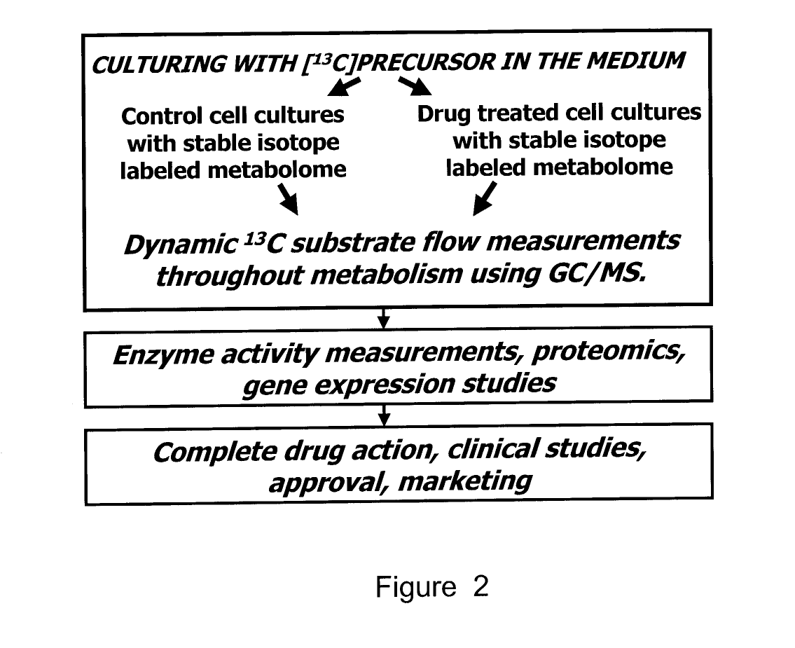

A method of doing business is disclosed and described here whereby drug candidates are efficiently analyzed, tested and brought to market. Compounds which are suspected of being useful as a drug are added to systems such as a cell culture and a determination is made on how the compound effects the system and in particular how the compound effects the system as compared to a substantially identical system without the compound or with a different concentration of the compound. The determination of effects on the system is made using a labeled substrate which may provide “heavy” non-radiating carbons. These carbons may be introduced into specific positions on a carbon chain of one or more key intermediates. This can reveal information about pathway substrate flux and re-distribution once the labeled molecules acted on by the system and are recovered. This is schematically shown in FIG. 2 where control drug free and drug treated cell cultures are incubated in the presence of [¹³C]precursor molecules, which may be [1,2-¹³C₂]glucose. The resulting ¹³C labeled metabolome is compared in terms of the activity of specific metabolic steps in control and drug treated cultures. Signaling, proteomic or genetic studies may follow to further explore biomarkers of diseases, characterize drug targets and determine efficacy.

The labeled molecules may be any molecules involved in cellular metabolism. For example the molecule may be ¹³C labeled glucose such as [1,2-¹³C₂]glucose. The label such as ¹³C can be followed in a living system such as determining how the ¹³C is made a part of the ribose sugar moiety of a nucleotide sequence—DNA and/or RNA.

Molecules involved in cellular metabolism are labeled at particular known positions for the invention. The labeled molecules are tracked and analyzed at one or more points in time as they move through and between metabolic cycles. Once precise information is gathered on how a labeled molecule is changed and distributed in a known system (e.g. a system comprised of a particular type of cells in a known cell culture medium) the system is characterized. System characteristics may change by adding a compound being developed as a possible pharmaceutically active drug. The manner in which the drug alters the way the labeled molecule is acted on by the system can then be used for drug characterization purposes. The changes observed relative to control drug free cultures in the system provide valuable information on factors such as mechanism of action, safety and efficacy of the drug tested.

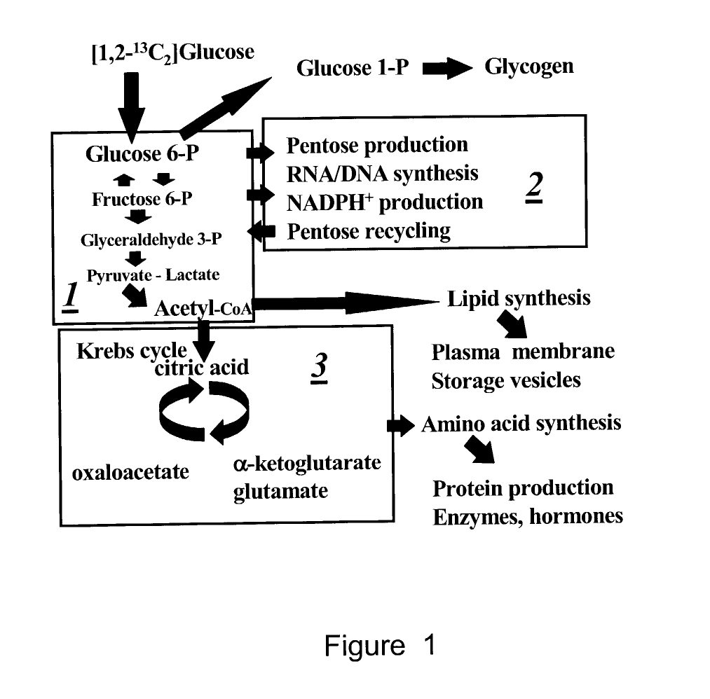

The method of the invention can not only predict but may exactly determine the metabolic steps involved in the formation of any glucose-based or glycogenic metabolite present in a living organism. This include glycogenic non-essential amino acids, nucleic acid ribose/deoxyribose and their bases, TCA cycle metabolites, phosphorylated glycolytic products, pentose cycle intermediates, glycogen, lactate, glutamate and non-essential fatty acids of the saturated and unsaturated kinds. These metabolites can be used to determine substrate flow and metabolic activity through glycolysis, glycogen synthesis, TCA cycle, pentose cycle, fatty acid synthesis and specific amino acid synthesis rates in mammalian cells, organisms and hosts. Known metabolic cycles of living organisms that can specifically be characterized by the [1,2-¹³C₂]glucose isotope tracer and dynamic metabolic profiling are shown in FIG. 1.

The present invention comprises the introduction of a precisely labeled general precursor molecule with a harmless non-radiating stable isotope on specific carbon positions. The label is allowed to distribute and rearrange with existing molecules and substrate pools in the cell. The next steps are to sample these pools, recover the stable isotope labeled intermediates and determine the positional distribution of the label. From the mass isotopomer information obtained, the invention provides information in the metabolic pathway flux and enzyme activities changes, the effects of genes and signaling pathways on metabolism. This information can be used to provide a comparison between healthy and diseased tissues and allows for the evaluation of drug treatments and other therapeutic interventions.

The system used can be any environment, which acts on and changes metabolic reactions. Thus, the system can be a chemical reaction, e.g. an enzymatically driven nucleotide replication reaction. At a more complex level the system can be a cell or cells in a cell culture medium. Any type of cell can be used including prokaryotic and eukaryotic cells, viruses or phages. In particular, mammalian cells such as human cells can be used. Plant as well as animal cells can be used. The system is preferably contained in a manner so as to reduce or eliminate unwanted influences.

At a still more complex level the system may be a tissue culture comprised of plant or animal tissue. The tissue may be, for example, tissue from a particular organ, which may be acted upon by the drug. By examining the effects of the drug on the system at a molecular level and comparing such to the system in the absence of the drug and/or to other known systems a great deal of information on the safety and efficacy of the drug can be ascertained.

At a still more complex level the system may be a multi-cellular organism. The organism may be a plant or an animal including a human. In one embodiment the system is a transgenic, non-human animal genetically engineered to have or be capable of getting a disease generally associated with humans. Testing drugs in transgenic animals could always provide some information on the safety and efficacy of the drug but can provide substantially more information via the present invention.

The system can be human or animal tissue hosting bacteria or viruses in order to study the metabolic particulars of bacterial replication or virus assembly in the presence of drugs, antibiotics in particular.

The system can be plant cells or bacteria hosting phages in culture or in vivo in order to study phage replication and assembly in primitive organisms.

### Glucose Intermediates Produced

The changing pattern of distribution of ¹³C carbons from [1,2-¹³C]glucose in intracellular metabolic intermediates can provide a measure of carbon flow toward the pentose cycle, glycolysis, direct glucose oxidation, TCA cycle and fatty acid synthesis, simultaneously. Metabolic profiling reveals specific flux changes in lactate, glutamate, nucleic acid ribose, palmitate and CO₂ during disease and health or during drug treatments or other interventions. Dynamic and comprehensive metabolic profiling thus indicates specific changes in glucose substrate utilization for macromolecule synthesis in living organisms, reveals the synthesis steps and provides information that can also be used for drug target development. The rationale for ¹³C labeling and interpretation of information gained by it during metabolic profiling are described below.

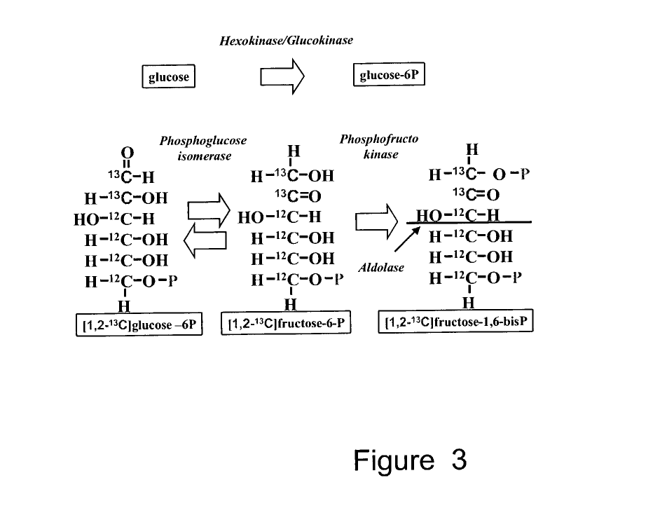

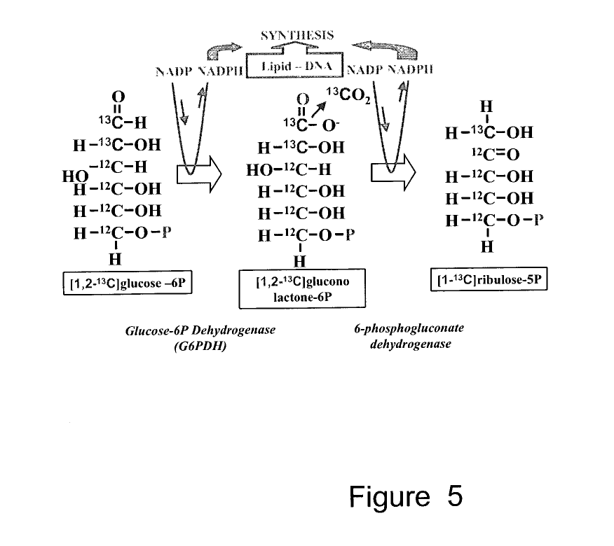

Glucose enters the cell as a broad and widely used substrate, or precursor of others, upon which it becomes activated (phosphorylated) as shown in FIGS. 1 and 3. Glucose provides carbons for the synthesis of glycogen, pentoses, nucleotides, glycolysis intermediates, TCA cycle metabolites, fatty acids, lactate, amino acids and many other molecules not discussed here. Box 1 in FIG. 1 shows glycolysis, box 2 shows the pentose cycle oxidative and nonoxidative branches and box 3 shows the tricarboxylic acid (TCA) cycle. ¹³C labeled glucose readily enters these metabolic cycles and labels metabolite pools. By its rearrangements specific substrate flow information is gathered by the invention. For example, ribose synthesis in the pentose cycle is possible through either the oxidative or the nonoxidative branches. Currently there are no label systems that can differentiate between the two branches, which produce chemically identical ribose. By the rearrangements of ¹³C in ribose we can differentiate between oxidative and nonoxidative pentose production which is a crucial metabolic process for nucleic acid synthesis, cell proliferation and cell differentiation as shown in FIGS. 5, 6 and 7.

In general, [1,2-¹³C₂]glucose metabolism produces four isotope-labeled intermediary metabolite species, also called mass isotopomers, m1: with one ¹³C substitution; m2: with two ¹³C substitutions; m3: with three ¹³C substitutions; and m4: with four ¹³C substitutions; which can reside in various positions in intermediary metabolites. These isotopomers are readily separated and measured using gas chromatography/mass spectrometry techniques described previously (Lee, W. N., Boros, L. G., Puigjaner, J., Bassilian, S., Lim, S., Cascante, M. (1998) Mass isotopomer study of the non-oxidative pathways of the pentose cycle with [1,2-¹³C₂]glucose. Am. J. Physiol. 274, E843-51; Lee, W. N., Edmond, J., Bassilian, S., Morrow, J. W. (1996) Mass isotopomer study of glutamine oxidation and synthesis in primary culture of astrocytes. Dev. Neurosci. 18, 469-77; and Lee, W. N., Byerley, L. O., Bassilian, S., Ajie, H. O., Clark, I., Edmond, J., Bergner, E. A. (1995) Isotopomer study of lipogenesis in human hepatoma cells in culture: contribution of carbon and hydrogen atoms from glucose. Anal. Biochem. 226, 100-12).

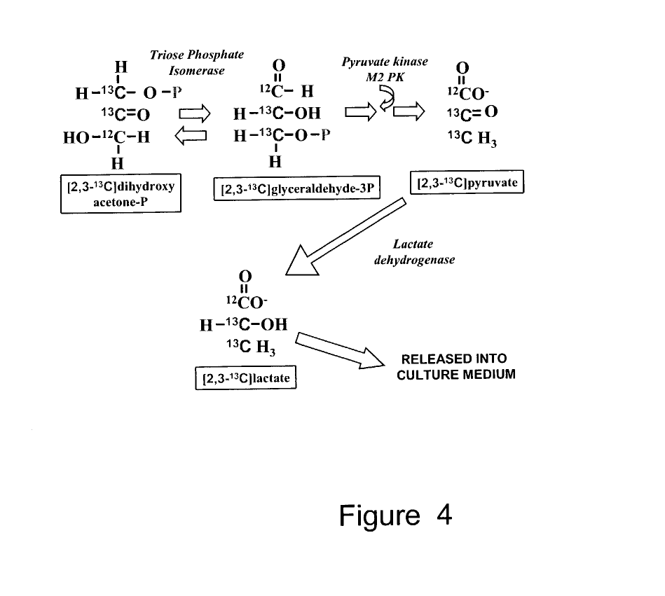

Lactate is the main three-carbon product of glycolysis and it is readily secreted into the cell culture medium. Accordingly, lactate can be utilized for the measurement of label incorporation into the three-carbon metabolite pool. The possible arrangements of ¹³C labels from [1,2-¹³C]glucose to lactate through glycolysis are shown in FIGS. 3 and 4.

Glucose oxidation through the pentose cycle on the other hand results in a loss of the first ¹³C of glucose that is shown in FIG. 5. During glucose oxidation ¹³CO₂ is also released which reflects glucose utilization for energy production in the pentose and TCA cycles. During metabolic profiling the method of the invention makes it possible to determine not only the amount of ¹³C isotope accumulation but also the positions of ¹³C labeled carbons within lactate. Those skilled in the art reading this disclosure will recognize that the ratio between m1 (recycled lactate from oxidized glucose via the oxidative branch of pentose cycle) and m2 (lactate produced by the Embden-Meyerhof-Parnas glycolytic pathway) is indicative of the activity of G6PDH and glucose recycling in the pentose cycle. A detailed description of the reactions and calculations can be found elsewhere (Lee, W. N., Boros, L. G., Puigjaner, J., Bassilian, S., Lim, S., Cascante, M. (1998) Mass isotopomer study of the non-oxidative pathways of the pentose cycle with [1,2-¹³C₂]glucose. Am. J. Physiol. 274, E843-51). Disease processes and drug treatments that affects direct glucose oxidation or glycolytic flux is expected to alter glucose label rearrangement in lactate.

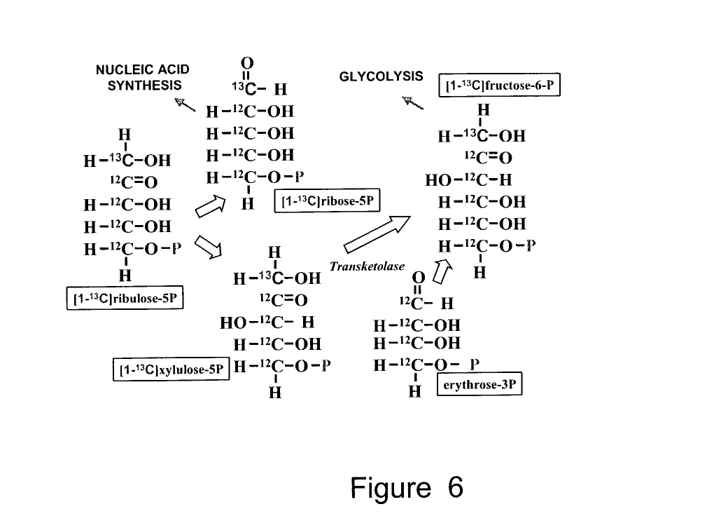

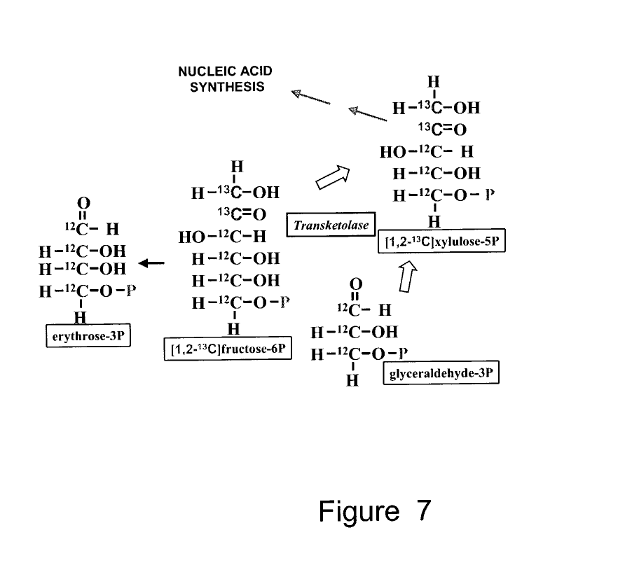

Ribose and deoxyribose are the building blocks of nucleotides and therefore ¹³C incorporation from glucose into RNA ribose or DNA deoxyribose indicates changes in nucleic acid synthesis rates through the respective branches of the pentose cycle. Singularly labeled ribose molecules (i.e., one ¹³C label) on the first carbon position (m1) represent ribose that is produced by direct glucose oxidation through G6PDH (FIG. 5). This ribose can either be incorporated into nucleic acid or returned to glycolysis as shown in FIG. 6. The alternative pathway for ribose synthesis is through the non-oxidative steps of the pentose cycle using glycolytic metabolites (FIG. 7). There is no net carbon loss throughout the non-oxidative steps of the pentose cycle; therefore, ribose molecules labeled on the first two carbon positions with ¹³C (m2) represent nucleic acid ribose synthesis through the non-oxidative route. The ratio between m1, m2, m3 and m4 of nucleic acid ribose/deoxyribose closely reflects the involvement of glucose oxidation and non-oxidative ribose synthesis in tumor cells. These reactions are effectively modulated by diseases and by various treatment modalities during de novo nucleic acid synthesis and cell growth.

¹³CO₂ release is a reliable marker of glucose oxidation (FIG. 5). ¹³CO₂ production from [1,2-¹³C]glucose takes place in both the pentose and TCA cycles and it is measured as part of the metabolic profiling processes to determine the rate of glucose oxidation in response to various drug therapies. Decreased glucose oxidation with increased glucose uptake is always a reliable sign of increased anabolism as seen in transformed cells.

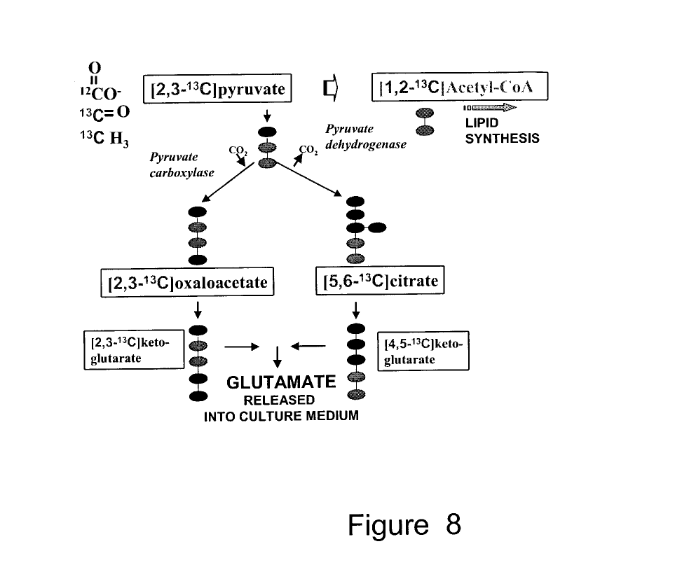

Glutamate, a non-essential amino acid, is partially produced from mitochondrial α-ketoglutarate, which is a central intermediate of the TCA cycle. Glutamate is readily released into the culture medium after synthesis, which represents one of the routes for glucose carbon utilization. Therefore, label incorporation from glucose into glutamate is a good indicator of TCA cycle anabolic metabolism for amino acid synthesis instead of glucose oxidation (FIG. 8).

Fatty acid synthesis is also strongly dependent on glucose carbons through the formation of acetyl-CoA via pyruvate dehydrogenase. The incorporation of ¹³C from [1,2-¹³C]glucose gives key information about the fraction of de novo lipogenesis in mammalian cells and about glucose carbon contribution to acetyl-CoA for fatty acid synthesis (FIG. 8). Many diseases and treatment modalities alter fatty acid synthesis, and changes in the flow of carbon toward fatty acid synthesis are important in cell growth control, differentiation, enzyme/hormone synthesis and new receptor formation.

The study of dynamic metabolic profiles using stable isotopes in cell cultures or in vivo reveals how the signaling and genetic events translate into metabolic processes, and also how substantially metabolic pathway flux changes influence cell growth. Effective therapeutics and drugs will alter carbon substrate flow in metabolic pathways in a desired manner, which can be reveled using our stable isotope based dynamic metabolic profiling technique. Therefore dynamic metabolic profiling is an excellent tool for screening potential new drugs to treat diseases.

### Information Obtained by Tracking Label

Molecules involved in cellular metabolism are labeled at particular known positions for the invention. The labeled molecules are tracked and analyzed at one or more points in time as they move through and between metabolic cycles. Once precise information is gathered on how a labeled molecule is changed and distributed in a known system (e.g. a system comprised of a particular type of cells in a known cell culture medium) the system is characterized. System characteristics may change by adding a compound being developed as a possible pharmaceutically active drug. The manner in which the drug alters the way the labeled molecule is acted on by the system can then be used for drug characterization purposes. The changes observed relative to control drug free cultures in the system may be used to provide valuable information on factors such as mechanism of action, safety and efficacy of the drug tested.

The method of the invention can be carried out with a number of different end results obtained. For example, the end result may be an evaluation of the effect of a compound such as a proposed pharmaceutically active drug on one or more metabolic pathways. The method is carried out by labeling precursor molecules which are preferably labeled with ¹³C isotope at a known position. The precursor molecule can be any molecule which normally contains a ¹²C. Further, 1, 2, 3, 4, 5, 6 or any number of ¹³C labels can be included within the precursor molecule. An example of a precursor molecule typically utilized in connection with the invention is a glucose molecule.

Once the precursor molecule such as the glucose molecule is labeled by having a ¹³C added in place of a ¹²C the precursor molecules are added to a changing test system. In terms of this system changing can be changing in any manner. However, it is typically a living system such as a cell, a group of cells in a cell culture or an animal which could be a human. Thus, changing does not mean that this system is completely different from one time to the next but rather that it is continuing to undergo biochemical reactions as are normally present within any living system.

After the labeled precursor molecules are added to the changing test system samples are extracted from the test system and molecules which incorporate the ¹³C label are analyzed. The molecules which are analyzed may be completely different molecules from the originally labeled ¹³C precursor molecules. The biochemical reactions of the changing test system may cause the ¹³C label to be added to other molecules, i.e. cause the normal carbon or ¹²C present in other molecules to be replaced by ¹³C. The analysis is carried out at a given point in time which can be referred to as a first point in time.

After the analysis is carried out the information is obtained and the obtained information is compared with information which may be obtained from a control system or compared to reference information which is previously obtained from a variety of sources including multiple control systems. In general, the control system is identical or substantially identical to the changing test system except that the test system has a single characteristic change. That single characteristic may, for example, be the addition of a compound which may be a proposed pharmaceutically active drug. The information is generally information such as changes in the molecular weight caused by the addition of the ¹³C molecule being analyzed. Such makes it possible to determine the position of the ¹³C within the molecule being analyzed. Thereby making it possible to determine the effects of the change such as changes caused by the pharmaceutically active drug on the system such as a change induced in a metabolic pathway of the system.

Preferably, samples are taken at multiple times which may be a second, a third, or fourth time which are each later in time from the first time and from each other. The samples taken at these different times are then analyzed and then compared with comparable information from a control system assayed at substantially the same points in time.

What the method of invention makes possible is the tracking of carbon atoms as they move through one or more different metabolic pathways of a changing system such as a cell culture. The precise position of the labeled carbon atom in a molecule can be tracked. The movement of that labeled carbon atom from one molecule to another at specific positions can reveal substantial amounts of information relating to the metabolic pathway of a living system. When that information is compared with a control system it is possible to obtain relatively precise information regarding the effect of a change such as an added proposed drug has on the system.

The system used can generally be any environment, which acts on and changes metabolic reactions and/or the molecule involved in such reactions. Thus, the system can be a chemical reaction, e.g. an enzymatically driven nucleotide replication reaction. At a more complex level the system can be a cell or cells in a cell culture medium. Any type of cell can be used including prokaryotic and eukaryotic cells, which may be alone or with viruses or phages. In particular, mammalian cells such as human cells can be used. Plant as well as animal cells can be used. The system is preferably contained in a manner so as to reduce or eliminate unwanted influences.

At a still more complex level the system may be a tissue culture comprised of plant or animal tissue. The tissue may be, for example, tissue from a particular organ, which may be acted upon by the drug. By examining the effects of the drug on the system at a molecular level and comparing such to the system in the absence of the drug and/or to other known systems a great deal of information on the safety and efficacy of the drug can be ascertained.

At a still more complex level the system may be a multi-cellular organism. The organism may be a plant or an animal including a human. In one embodiment the system is a transgenic, non-human animal genetically engineered to have or be capable of getting a disease generally associated with humans. Testing drugs in transgenic animals could always provide some information on the safety and efficacy of the drug but can provide substantially more information via the present invention.

The system can be human or animal tissue hosting bacteria or viruses in order to study the metabolic particulars of bacterial replication or virus assembly in the presence of any type of pharmaceutically active drug, e.g. oncology and antibiotics in particular.

The system can be plant cells or bacteria hosting phages in culture or in vivo in order to study phage replication and assembly in primitive organisms.

### From Metabolic Profiling to Marketing

Methods of using metabolic profiling are disclosed and described above and further exemplified below. Per these methods molecules such as glucose are labeled such as with ¹³C and the label is tracked through one or more metabolic pathways in a system such as a cell culture (FIG. 2). The tracking is carried out while the system is being subjected to a test influence which may be sound, light, heat, pH, etc or combinations thereof, but is generally a compound being tested for use as a drug. The results obtained when the test influence or compound is present is compared to a standard or control. The standard may be a standard independently developed by others which shows how the system operates in the absence of the test influence. A control may be an identical or substantially identical system operated in the absence of the test influence e.g. without the compound present. The control may be run contemporaneously with the test run or may have been run earlier by those carrying out the test run or by others to develop a standard. The metabolic profiling methodology is a significant aspect of the invention and information obtained from such allows for making a more informed discussion on which compounds are selected for additional testing.

The additional testing may take a variety of forms. However, with suspect to drugs in the U.S. the testing generally results in what is referred to as Phase I, Phase II and Phase III trials. Such testing can be enormously expensive and may cost tens or even hundreds of millions of dollars. The testing may be carried out directly by the entity performing the metabolic profiling methodology or could be carried out by others independently or on behalf of those doing the original metabolic profiling.

The testing is generally completed once evidence has been provided to show that the compound can act as a safe and effective treatment for a disease. For example, Phase III trials are completed in the U.S., and at that point an application for approval to market the drug is made to the appropriate government agency—in the U.S. the Food and Drug Administration or FDA. In the U.S. the application for approval may be referred to as a New Drug Application or NDA. The application (e.g. NDA) is reviewed by the government agency (e.g. FDA) and if a sufficient showing of safety and efficacy is made approval to market is granted. The drug may then be sold by the same or a different entity from the entity carrying out all or some of the other steps of the overall business method.

From the above it will be understood that an aspect of the invention comprises a method of doing business comprising (a) the metabolic profiling to determine which compound should be tested further; (b) further testing which may include clinical trials; (c) applying for and obtaining governmental approval to market the compound as a drug; and (d) marketing the drug. The steps together provide the overall business method and may be carried out by the same entity or by several entities all on behalf of the entity eventually marketing the drug.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

The details of how the invention can be carried out can be better understood by reference to the figures. For example, FIG. 3 shows the structure of a preferred embodiment of a labeled glucose molecule along with possible rearrangements of ¹³C in various metabolites of glycolysis using [1,2-¹³C₂]glucose as the single tracer. Glucose activation via hexokinase/glucokinase and the formation of fructose-1,6-bis phosphate maintain the ¹³C labeled carbons in the 1^{st} and 2^{nd} positions. ¹³C-labeled carbon positions derived from [1,2-¹³C₂]glucose are shown by the “13” superscript, while ¹²C native-labeled carbon positions are shown by the “12” superscript. Participating enzymes are italicized in all of the figures. To carry out the invention [1, 2-¹³C₂]glucose is added to a cellular system and tracked through a biochemical pathway such as the pentose cycle, glycogen synthesis, tricarboxylic acid cycle, glycolysis, lactate synthesis, glutamate production, fatty acid synthesis and nucleic acid ribose and deoxyribose synthesis pathways by themselves or simultaneously.

### Example 2

In addition to labeling glucose as shown in FIG. 3, it is possible to label glucose at other positions and/or to label other molecules such as [2,3-¹³C]dihydroxy acetone-P or to continue to track the molecule of [2,3-¹³C₂]dihydroxy acetone-P created in the reaction show in FIG. 3. FIG. 4 shows the structure of the labeled compounds involved in the formation of [2,3-¹³C₂]lactate through the Embden-Meyerhoff-Parnas pathway. The production of three-carbon metabolites by aldolase (as shown in FIG. 3), glyceraldehyde and dihdroxy acetone phosphates transfers the labeled carbons into the 2^{nd} and 3^{rd} positions of glyceraldehyde. There are no subsequent positional changes in terms of ¹³C labeling by triose phosphate isomerase in the three-carbon metabolite pool that undergoes glycolysis, resulting in the release of lactate.

### Example 3

The labeled glucose as shown in FIG. 3 can be acted on differently as the reactions of FIG. 5 show. FIG. 5 shows the structure of compounds involved in the rearrangement of ¹³C in pentose cycle metabolites due to direct glucose oxidation. The loss of the first labeled carbon of glucose due to direct oxidation produces ribulose molecules that are labeled only on the first position with ¹³C. During the oxidation of glucose ¹³CO₂ is released, which can easily be detected using isotope ratio mass spectrometry (IRMS). Reducing equivalent NADP⁺ is also produced that can be used in lipid synthesis, DNA nucleotide production or to maintain reductive/oxidative reactions throughout metabolism.

### Example 4

The [1-¹³C]ribulose-5P molecule shown on the far right of FIG. 5 can be produced and labeled. Alternatively, the molecule can be tracked through one or more additional reactions. The molecule on the far right of FIG. 5 is on the far left of FIG. 6 which shows the structure of compounds involved in the formation of [1-¹³C]ribose-5P in the non-oxidative pentose cycle after glucose oxidation. The nonoxidative steps of the pentose cycle generate a number of intermediates that can be used for nucleic acid synthesis (ribose-5P, as seen in proliferating cells) or recycled back to glycolysis (glyceraldehydes-P and fructose-P, as seen in non-proliferating/resting cells).

### Example 5

Nucleic acid synthesis is essential for cell replication and FIG. 7 shows the structure of compounds involved in the formation of [1,2-¹³C₂]ribose through the non-oxidative reactions of the pentose cycle. Rapidly proliferating cells are able to synthesize ribose-5P via non-oxidative pentose cycle reactions. This process allows the unrestrained production of ribose-5P, independent of available NADP, a phenomenon observed in response to cell transforming agents. Increased non-oxidative synthesis of ribose from glucose deprives mammalian cells of reducing equivalents. Although a great proliferating potential is engendered, reductive synthesis, differentiation, normal cell morphology and functions are diminished.

### Example 6

A compound such as pyruvate can also be labeled as shown FIG. 8 which shows [2,3-¹³C₂]pyruvate. FIG. 8 shows the structure of compounds involved in the formation of ¹³C labeled acetyl-CoA and glutamate through pyruvate dehydrogenase and pyruvate carboxylase in the TCA cycle. Glucose carbons readily label TCA cycle metabolites and fatty acids because the first two carbons of glucose form the acetate molecules that enter the TCA cycle and lipid synthesis pathways.

The examples 1-6 described above and schematically shown in FIGS. 3-8 demonstrate that the invention can be used in a wide variety of situations. Specifically, different molecules can be labeled and tracked for different periods of time through different metabolic cycles. It is preferable to label molecules that are well known to be acted on in a particular manner in a well-known and well-characterized metabolic cycle.

### Informational Database

Genetic and signaling events in cells translate into metabolic changes that determine the function and phenotype of the cell. Changes in genetic, signaling and protein synthesis pathways can readily be revealed using molecular and proteomics technologies. The dynamic metabolic profiling technology provided here supplements these existing technologies by investigating changes through a specific stable isotope labeled metabolome. The invention can be carried out in a manner so as to develop a large database of the dynamic metabolome of various cell types. The information is then searched and used during drug design and target discovery in disease and health as a database, where sufficient patterns and pathway flux profiles are stored.

As the method of the invention is used and accepted it is expected to become the main metabolic profiling method for industrial and academic drug target design and new drug discovery processes. The invention makes it possible to set up “dynamic metabolic profiles” substrate utilization and distribution databases for various disease processes, signaling mechanisms, gene mutations and drug actions. Such a database can be searched for matching metabolic profiles by the industry or academic community in order to determine certain expected drug effects, signaling mechanisms and genetic events.

The metabolic profiles induced by cell transforming agents such as transforming growth factor beta (TGF-beta) and the organophosphate pesticide isofenphos has been determined that indicates intense nucleic acid ribose synthesis through the non-oxidative steps of the pentose cycle and increased cell proliferation rates.

The metabolic profiles of anticancer compounds such as Avemar and Gleevec indicate that effective tumor growth control can be achieved when glucose activation, oxidative and non-oxidative ribose synthesis from glucose are inhibited.

Increased fatty acid synthesis increases cell differentiation in response to Avemar treatment.

These metabolic profiles can already be used as part of the stable isotope labeled metabolome database for additional drug effects and signaling mechanisms.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of doing business, comprising the steps of:
using a ¹³C labeled molecule to determine information on how carbon atoms of the labeled molecule are repositioned when acted on in a living system;
analyzing the determined information and applying the analysis to select a lead compound for clinical trials;
conducting clinical trials on the selected lead compound; and
marketing the lead compound as a pharmaceutical drug after completing the clinical trials and obtaining governmental approval.

2. The method of claim 1, wherein the living system comprises a living cell.

3. The method of claim 1, wherein the living system comprises a plurality of living cells in a cell culture.

4. The method of claim 1, wherein the living system comprises living tissue.

5. The method of claim 1, wherein the living system comprises a multi-cellular organism.

6. The method of claim 1, wherein the ¹³C labeled molecule is ¹³C labeled glucose.

7. The method of claim 6, wherein the ¹³C labeled glucose is chosen from [1,2-¹³C₂] glucose, [1,2,5,6-¹³C₄] glucose and [5,6-¹³C₂] glucose.

8. The method of claim 1, wherein the ¹³C labeled molecule is [1,2-¹³C₂] glucose and information is determined by comparing data obtained when the living system is in contact with a candidate compound to data obtained when the living system is not in contact with the candidate compound.

9. The method of claim 1, wherein the determining information comprises determining molecular weight of ¹³C labeled molecules acted on by the living system.

10. The method of claim 8, wherein the determining information comprises determining positions of at least two carbon atoms of ¹³C labeled molecules acted on by the living system and tracking changes in those positions as the labeled molecule moves through a metabolic pathway.

11. The method of claim 8, wherein the determined information is analyzed to obtain data on how the candidate compound changes metabolic pathways of the living system involved in assembling components of a new living system.

12. The method of claim 1, wherein information is determined on molecular weight and ¹³C labeled carbon positions of the ¹³C labeled molecule acted on by both a control living system and a living system acted on by a candidate compound.

13. The method of claim 1, wherein the using of a ¹³C labeled molecule to determine information and the analyzing of the determined information is carried out by
adding ¹³C labeled molecules to a system which labels change the molecular weight of molecules in the system which incorporated the ¹³C label;
analyzing molecules in the system to determine changes to molecular weights;
analyzing molecules in the system to determine positions of ¹²C and ¹³C carbons;
comparing determined changes to molecular weights and ¹³C carbon positions in a control versus a compound treated system.

14. The method of claim 13, wherein the comparing is carried out to reveal effects of the compound on a specific metabolic pathway of the system; and
wherein the system is comprised of organisms chosen from cells and viruses which system is producing new organisms and the metabolic pathway is one which is involved in production of the new organisms; and
wherein the ¹³C labeled molecules are ¹³C labeled glucose chosen from [1,2-¹³C₂] glucose, [1,2,5,6-¹³C₄]glucose, [5,6-¹³C₂]glucose.

15. The method of claim 13, wherein the system is chosen from a plurality of living cells in a cell culture, living tissue, a multi-cellular organism, bacteria cells, plant cells, bacteria hosting phages, cells hosting viruses and cells hosting virus particles.

16. The method of claim 13, further comprising:
separating from the system molecules which have incorporated ¹³C label
wherein the separating is carried out by a means chosen from centrifugation, physical/chemical purification, chemical derivatization, liquid chromatography and gas chromatography; and
wherein the analyzing is carried out by spectrometry selected from mass spectrometry and nuclear magnetic resonance.

17. A method of doing business, comprising the steps of:
determining a metabolic step involved in the formation of a glucose based metabolite of a living organism by:
adding ¹³C labeled glucose to a living cellular test system in the presence of a test compound;
allowing the test compound to interact with the test system for a given period of time under known conditions;
separating ¹³C labeled molecules away from the test system;
analyzing the separated ¹³C labeled molecules;
comparing analysis results obtained from the test system with results obtained from a known system;
using results of the comparing to determine if the test compound should be tested further;
conducting further testing on the test compound; and
marketing the test compound.

18. The method of claim 17, wherein the living cellular system is chosen from a known cell culture grown on known cellular nutrients, a transgenic mouse, a genetically normal wild type animal, and a human.

19. A method, comprising the steps of:
adding ¹³C labeled molecules to a test system which test system acts on the molecules and which ¹³C label changes molecule weights of molecules which the label is added to;
analyzing ¹³C labeled molecules in the system after the system has acted on the ¹³C labeled molecule added to the system and obtaining information from the analyzing;
comparing the information obtained with information on a known system.

20. The method of claim 19, wherein the known system is a control system substantially identical to the test system except for a compound added to the test system.

21. A method of determining the specific metabolic steps involved in the formation of a glucose based metabolite of a living organism, comprising the steps of:
adding ¹³C labeled glucose to a living cellular system;
allowing the system to act on the labeled glucose for a given period of time under known conditions;
separating ¹³C labeled molecules away from the system;
analyzing the separated ¹³C labeled molecules; and
comparing analysis results to analysis results obtained from a known system.

22. A method, comprising the step of:
adding ¹³C labeled molecules to a system which ¹³C label replaces ¹²C in molecules increasing the molecular weight of the molecules where the ¹³C replaces a ¹²C;
analyzing molecules incorporating ¹³C labels to determine changes to molecular weights relative to when the molecule was comprised of ¹²C;
analyzing ¹³C labeled molecules to determine the positions of ¹²C and ¹³C labeled carbons;
comparing determined changes to molecular weights and ¹³C labeled carbon positions in control versus a drug treated system in order to reveal specific drug action on molecules of the system.

23. The method of claim 22, wherein the comparing is a comparing of changes to molecular weights and ¹³C labeled carbon positions in an organism chosen from a bacteria, cell, virus and phage to reveal metabolic pathways involved in the assembly of a progeny of the organism.

24. A method, comprising the steps of:
labeling precursor molecules with a ¹³C isotope at a known position;
adding the labeled precursor molecules to a changing test system;
analyzing molecules in the test system which molecules have incorporated the ¹³C label, wherein the analyzing is carried out at a first point in time;
comparing information obtained from the analyzing with information chosen from a control system information and reference information.

25. The method of claim 24, wherein the comparing of information is used to determine how a metabolic pathway of the test system is changed relative to the control system information or reference information.

26. The method of claim 24 further comprising:
analyzing molecules in the test system which have incorporated the ¹³C label at a second point in time after the first point in time.
